(19) United States
(12) Patent Application Publication
(10) Pub. No.: US 2015/0327775 A1
(43) Pub. Date: Nov. 19, 2015

(54) MEDICAL EXAMINATION APPARATUS

(71) Applicant: Vandette Carter, Yorktown Heights, NY (US)

(72) Inventor: Vandette Carter, Yorktown Heights, NY (US)

(73) Assignee: PARACE, LLC, Bronx, NY (US)

(21) Appl. No.: 14/117,565

(22) PCT Filed: May 14, 2012
(86) PCT No.: PCT/US12/37857

§ 371 Date: Jul. 28, 2015

Related U.S. Application Data
(63) Continuation of application No. 13/160,493, filed on Jun. 14, 2011, now abandoned.
(60) Provisional application No. 61/518,852, filed on May 13, 2011.
(60) Provisional application No. 61/571,209, filed on Jun. 23, 2011.

(51) Int. Cl. A61B 5/0205; A61B 5/0408; A61B 3/12; A61B 5/1455; A61B 8/00; A61B 1/227; A61B 7/04; A61B 5/00

(52) U.S. Cl. CPC A61B 5/0205; A61B 7/04; A61B 5/04085; A61B 5/741; A61B 5/14552; A61B 8/4416; A61B 5/0077; A61B 1/227; A61B 3/12; A61B 5/0006; A61B 5/7445; A61B 5/0013; A61B 8/56; A61B 5/6898; A61B 2560/0443; A61B 2562/0209; A61B 2562/16

(57) ABSTRACT

An apparatus to enable health care providers to perform a number of diagnostic procedures, including listening to the heart, obtaining electrocardiograms, sonograms, ocular and otoscopic exams; audio and video functions as well as mobile phone capabilities, not limited to voice and text messaging. A pocket size instrument displays an ECG tracing on its screen simultaneously upon auscultation of the heart. The screen allows for audio-video display, Spo2 and touch screen functions. Wireless broadband technology may provide a closed circuit or global interface for real-time wireless transmission of acquired data, audio, photos and videos. Many common non-invasive examinations and tests can be performed without the need for a multiplicity of devices.

### BACKGROUND OF THE INVENTION

1. Field of the Invention

The present invention relates to apparatus that may be used to examine a living body. More particularly, it relates to apparatus that may be used for listening for body sounds and for receiving electrical signals and vital signs from the body. The invention also relates to a communication device for sending and receiving audio and image and video signals over a network or similar devices.

2. Background Art

The stethoscope since its perfection in 1850's by a New York Physician, Dr. George Cammann, has remained relatively unchanged in the last two centuries. Not much has been done or modified from its basic design and function since then. The stethoscope, as it is to date, is greatly limited by what it can do. In the examination of the heart, only sound can be appreciated. While helpful in certain limited clinical situation, sound tells the medical practitioner very little about the true characteristic of the heart, such as its conduction or electrical activities, ejection fraction, wall muscle size, damages or its true physical state.

The general shape of the stethoscope underwent some minor improvements. However, what has remained constant for the past 150 years is the appearance. There is probably a good reason for this. Besides the white coat that is so symbolic of a doctor, it is the stethoscope instrument that easily identifies a health care professional. The stethoscope has been revered as a symbol of the medical profession and more specifically for the health care provider. In some ways, in terms of its symbolism, the shape of the stethoscope can be likened to the gavel a judge uses.

Due to the cumbersome nature of bulky machines, complex lead arrangements, or the need for a technician of obtaining an ECG in the general population, one is not routinely done in common practice.

For the most part, an EKG is reserved for an age specific population or for people with established or suspected cardiac illnesses. In both cases, the study is usually formerly requested and time is the factor that most clinicians have to deal with.

Further, clinicians are required to perform more patients evaluation in a fraction of time than was allotted nearly half a century ago. To enhance this requirement with efficiency, numerous bedside instruments are made to be interchangeable with the third part of the apparatus. For instance, the third part headset maybe interchanged with an otoscope probe (digital), while the image of the ear canal and ear drum is displayed on the first part's screen.

### SUMMARY OF THE INVENTION

It is an object of the invention to incorporate technological advances while preserving the basic design and functionality of the traditional stethoscope.

It is another object of the invention to provide an apparatus for obtaining an EKG rhythm quickly and efficiently.

It is yet another object of the invention to provide an apparatus that avoids the need for a multiplicity of electrical leads that are difficult to manage, and may become tangled.

It is still another object of the invention to incorporate a wireless phone technology, pager, pulse oximeter, recordable and downloadable audio/video, internet access, capture and upload photo and video using the apparatus, in a manner consistent with the advancement in the field, such as digital.

These objects and others are achieved in accordance with the invention in an apparatus for determining a condition of a living body, comprising a first portion for acquiring acoustic signals from said body, said first potion having removeable electrodes for acquiring electric signals from said body; a second portion comprising an elongate housing having conduits for conducting signals from said first portion; and a third portion comprising a headset for listening to said acoustic signals; wherein said third portion is removeably connectable to said second portion. The apparatus may further comprise a communication link between said second portion and said third portion for transferring said signals when said third portion is not connected to said second portion. The communication link is preferably a broadband communications link. It is preferably a wireless communication link.

Moreover the invention also relates to an apparatus with multiple interchangeable third parts instruments that maybe displayed graphically on the first part. It also relates to an apparatus, which have the capability of examining a body with ultrasound, pulse-oximeter, camera, video, otoscope, opthalmoscope and listening to audio stored within the device or from an external source.

The apparatus may further comprise a plurality of EKG electrical cable wires, coupled to electrodes. Said cable wires maybe secured inside of the cavity of second portion using Velcro or magnet. Said second part will remain flexible when the cable wires are secured therein. The practitioner may use the cable wires in a traditional method to obtain EKG signals.

The first portion may further comprise a display for providing a visual indication representative of at least one of said signals obtained from the first part using one of a removable electrode, Doppler probe, sono probe or digital data obtained from part #3 interchangeable instruments. The apparatus may be in combination with an external display coupled to said apparatus with a broadband wireless real-time connection for data originating in said apparatus or interchangeable third part.

The first portion may further comprise an ultrasonic transducer usable for performing an ultrasonic examination of at least a portion of said body. The first portion may also have at least one replaceable electrode that is coated with an element or compound such as silver chloride when copper or another metal is used to form the mass of the electrode of part #1. It should be noted that the silver chloride may erode off of the copper, exposing it and decreasing its electrical detection conductivity. The user will then easily replace said electrode/s with a tool provided. Electrodes maybe also be made of pure silver when copper and combination of silver or silver chloride coating is not used.

The apparatus may be in combination with an external display coupled to said apparatus with a broadband wireless real-time connection for data originating in said apparatus, said data being derived from at least one of a sonogram, and an electrocardiogram. The second portion may be configured with an elongate cavity to house a set of traditional cable wires/electrodes in an concealed manner. The first or second part may house spo2 optical sensor to acquire pulse oximeter (oxygen saturation content) as well as blood sugar level.

The first portion, the second portion and the third portion of the apparatus may each comprise at least one electrode for receiving electrical signals from said body. The first portion may also have at least one camera lens to acquire a photo or video image.

The apparatus may further comprise at least one adjustable light source on said first portion—preferably two that converges at a focal point.

The first portion may comprise a diaphragm coupled to an electrical microphone to detect low to midrange frequency heart, lungs and abdominal sounds in the average spectral range of 20 hz to 2000 hz. Collectively the apparatus can have a first part, a second part, and a third part headset comprising a first earpiece and a second earpiece The first portion microphone sensor conveys detectable body sounds to the third part via electrical conduits traversing the second part. A speaker is housed in the third part to broadcast sound to the respective left and right earpieces via internal acoustic pathways.

The first portion may be releasably connected to said second portion but preferably fixed for ease of use, and said second portion will be releasably connected to and said third portion to permit for multiple interchangeable instruments substitutions. The invention is also directed to a method for obtaining an electrocardiogram, by using the apparatus as described herein.

With respect to the photo and video, a camera lens maybe situated anywhere on the device for the purpose of capturing imagery, but preferably inside the connection of the third portion and the second portion so that the lens maybe secured from damage. The acquired data may be stored in the device or wirelessly shared or upload via USB to a central terminal.

In other applications of the apparatus, the user may receive/send a wireless phone call directly from the device while in transit anywhere within or outside of a hospital clinic facility. User may not have to interrupt or wait indefinitely for a consult or reach another physician etc. Multimedia alphanumeric text messaging, maybe send or received using the apparatus; internet connection and audio/learning digital files can be transferred (downloaded) into the device and playback at a later time or during a live conferencing via satellite or close broadband internet access.

An attachable pulse oximeter can be secured to the second portion of the apparatus, into the USB connection when the third portion is removed from the second portion to obtain a patient's oxygen saturation status non-invasively. Similarly, numerous other devices/instruments maybe attached to the second part when the third part (headset) is removed. Likewise a speaker maybe connected to said second part to allow the broadcast of sounds in the immediate vicinity so that medical students or professional colleagues maybe able to hear the sounds originating from a patient.

In another embodiment, the pulse oximeter is built into the device and results displayed on the color screen of first part. Similar operation maybe performed to obtain glucose level using an attachable glucometer to said second part, all of which can be synced to the main PCB of the first portion via the USB connection port accessible on the second portion (only visible when said third part is removed) A set of release buttons are positioned on the sides of part #2, to allow for the removal of the various instruments of the third portion.

There are many other additional technical details that may be included which will occur to those skilled in the art after reading the present description. For example, the head of the first portion may be removeably connected to the second portion so that they can be separately replaced or serviced, if necessary. Further, a rechargeable battery may be placed in housing of the head, which may be recharged by conduction means or standard outlet form using 220 or 110 v.

A detachable speaker can be interchanged with the third part to provide audio broadcast to third party listeners such as medical or nursing students. The ultrasonic transducer 40 may operate in any one of several modes including A scan, B scan, Doppler or so called “M” mode. A sound sensor microphone may transmit broadband wireless sound to third party listeners and to the third portion of the device via electrical wires and speakers housed into the third portion. Digital communication with the communication modules may be compatible with all digital forms of data transmission, display and analysis. The option of downloading the images to a mainframe computer/printer with interconnecting digital ports is available. Also the wireless transfer of data either by phone wires, cable wires, satellite signals or broadband network such as UWB, Bluetooth, Wi-Fi, WiMax, EDGE or 3G are all possible. While the apparatus has been described with a stethoscope portion with acoustic functions and pathways, it will be understood that an electronic stethoscope having a sound/vibration sensor or transducer for providing an electrical signal to amplify, and earpieces responsive to the amplified signal for producing sound for diagnostic or monitoring purposes, may also be used. Finally, one or more microprocessors may be included to process and store data.

Sonogram technology has been adapted into the stethoscope device to investigate all forms of pathologies within the abdomen and heart. As before, the sonogram probe maybe placed in the first portion, or made to be the replaceable third portion.

Likewise multiple probes (common instruments such as opthalmoscope) maybe engineered to be interchangeable as the third portion or built into the first portion.

Numerous other instruments such as pulse oximeter, camera, pager, can all be integrated into the apparatus as a removable/interchangeable segment or build into parts 1 or 2 and linked directly to the main circuitry found in part #1

The apparatus in accordance with the invention provides the following advantages:

1. All of the benefits of an EKG, phone, internet, audio-video recording/playback, sonogram and a diagnostic light source or sources are brought to the “finger tips” of a clinician.

2. Precious time needed to provide an EKG is reduced from hours to virtually seconds.

3. Lives can be preserved when pathologies in the heart, vascular systems, can be detected upon an initial encounter.

4. The delivery of life saving care to patients is expedited.

5. Human resources needed to perform sonogram and EKG studies are conserved.

6. The bulky machine and cumbersome, often tangled wires associated with a twelve lead study are eliminated.

7. An excellent and inexpensive screening and diagnostic tool for the chest and abdomen are provided.

8. The most ubiquitous and some would say the most indispensable medical tool (the stethoscope) is modernized and enhanced.

9. Cardiac examination protocols may be revolutionized.

10. Doctors and medical personnel are given the confidence to do more with one comprehensive device.

11. Wireless phone technology is made available.

12. Audio, photo and video in digital or more advanced form is available.

The invention also relates to a device that augments one or more interchangeable units to the main body of the stethoscope. Each of the individual units can be independently operable or made to be codependent when linked physically or wireless to the main body of the stethoscope or to an external display.

### BRIEF DESCRIPTION OF THE DRAWINGS

The foregoing aspects and other features of the present invention are explained in the following description, taken in connection with the accompanying drawings, wherein:

FIG. 1 is a perspective view of an embodiment of the apparatus.

FIG. 2 is side elevational view of the apparatus of FIG. 1.

FIG. 3 is a top or plan view of the apparatus of FIG. 1.

FIG. 4 is a bottom view of the apparatus of FIG. 1.

FIG. 5 is an electrical block diagram of the embodiment.

FIG. 6 is a functional electrical diagram of the embodiment.

FIG. 7 illustrates a possible approach for the arrangement of the relationship between various images and data, on a display screen.

FIG. 8 is an embodiment having a removable plate or module which may have different electrode configurations.

FIG. 9A and FIG. 9B illustrate an embodiment having replaceable screw-in electrodes which are coated with an element or compound (AgCl or Ag), when the mass is made of copper.

FIG. 10 is a perspective view of an earpiece for use with the embodiment of FIG. 1 to FIG. 4.

FIG. 11 is a schematic, partial cross-section of an embodiment of an earpiece.

FIG. 12 is a schematic, cross-section of an embodiment of an earpiece.

### DETAILED DESCRIPTION OF THE PREFERRED EMBODIMENTS

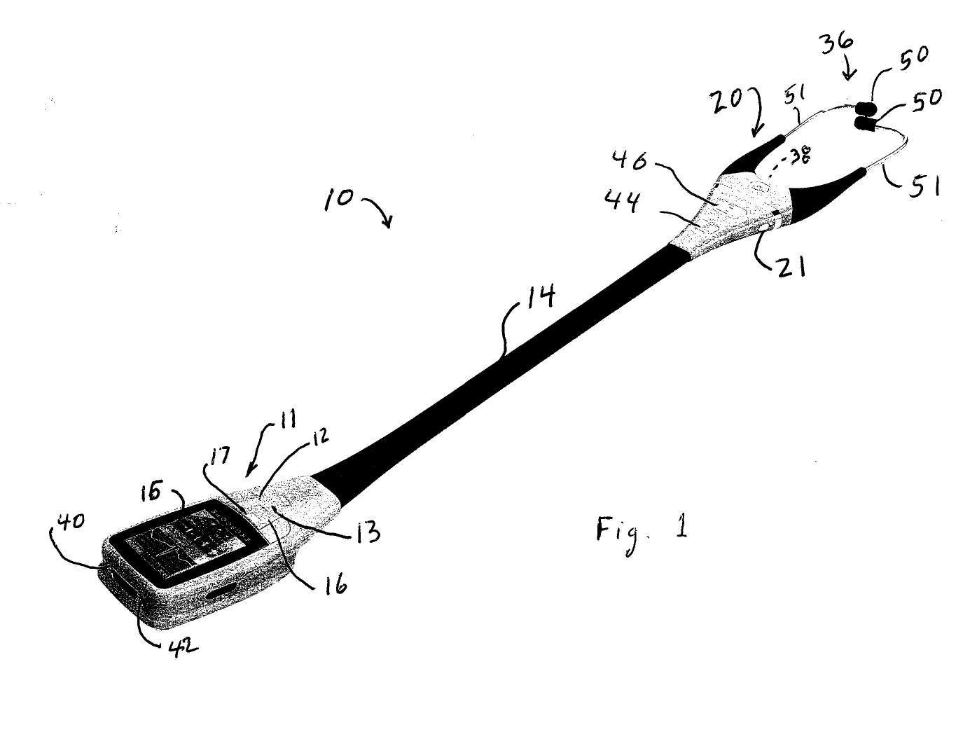

Referring to FIG. 1, there is shown a perspective view of an embodiment in the form of an apparatus 10. Although the description is with reference to the embodiment shown in the drawings, it should be understood that the presently described apparatus can be embodied in many alternate forms or embodiments. In addition, any suitable size, shape or type of elements or materials could be used.

In order to assist in an understanding of the embodiment described herein, reference is made to my prior issued U.S. Pat. No. 7,300,406, which is incorporated herein in its entirety.

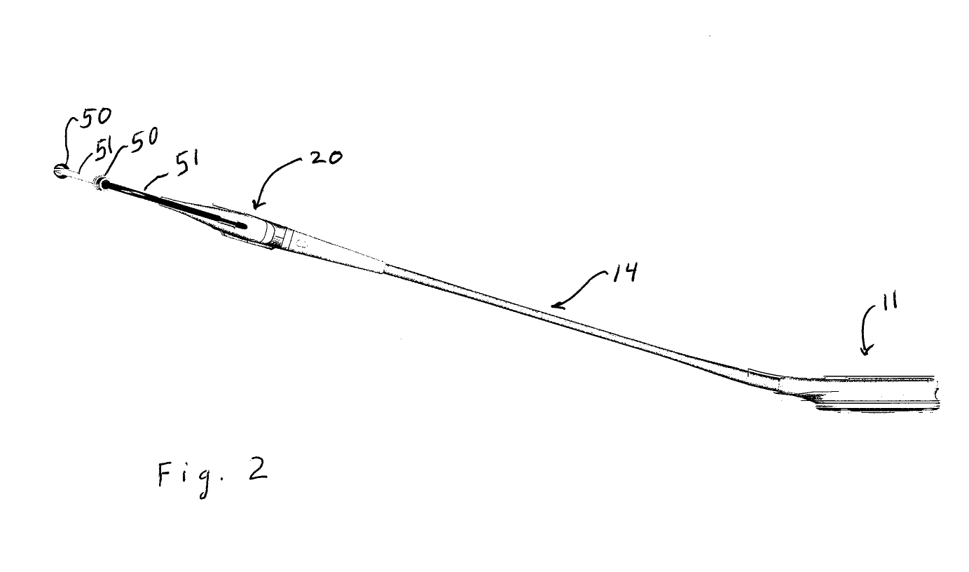

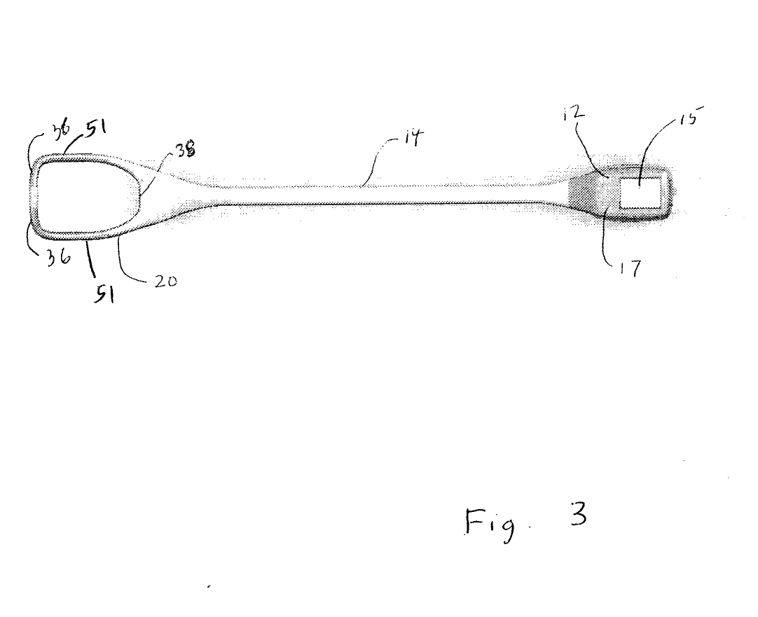

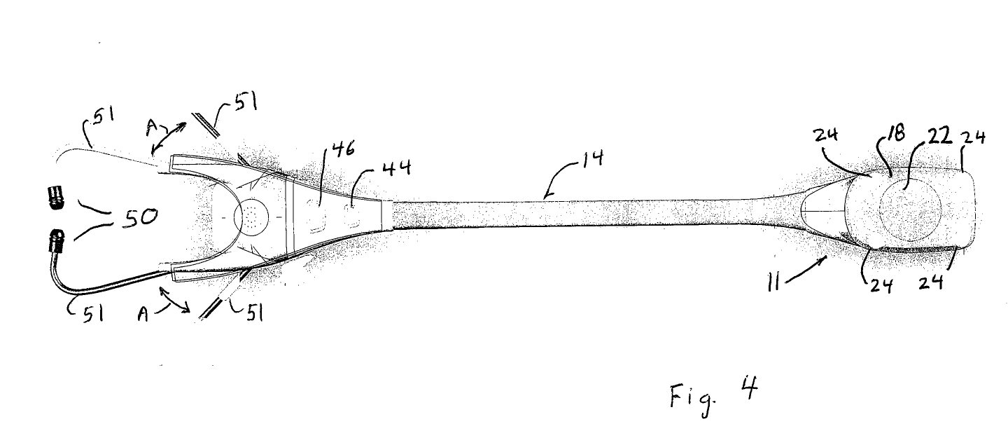

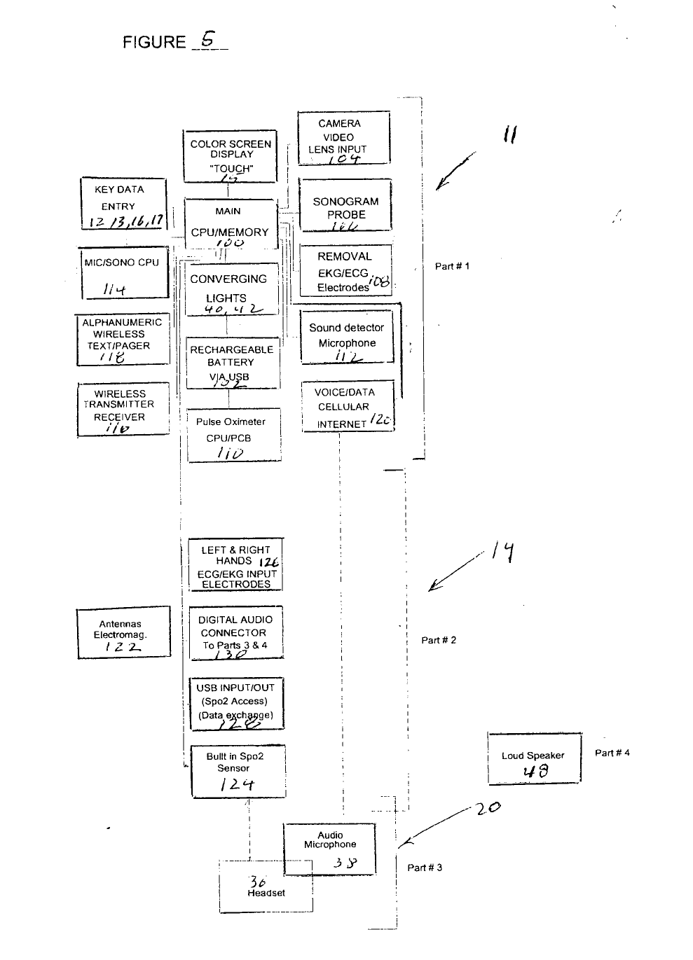

Referring to FIGS. 1 to 4, a first part of the apparatus 10, includes a head 11, which has a display screen 16; and entry buttons 12, 13, 16, and 17. A patient contact surface 18 includes a sound diaphragm 22 and ECG removable electrodes 24 (FIG. 4). Two light sources 40 and 42 (which are preferably light emitting diodes) project overlapping beams of light (not shown) as an aid to examination of a patient's body. A miniature camera 104 (FIG. 5), which may be of the CCD type, may be positioned on head 11 to record images of an illuminated region of the body, or of the body generally. An ultrasonic transducer (not shown), for assisting in examination of the body may also be placed on head 11, but preferably interchangeable with the third portion (headset)—not shown.

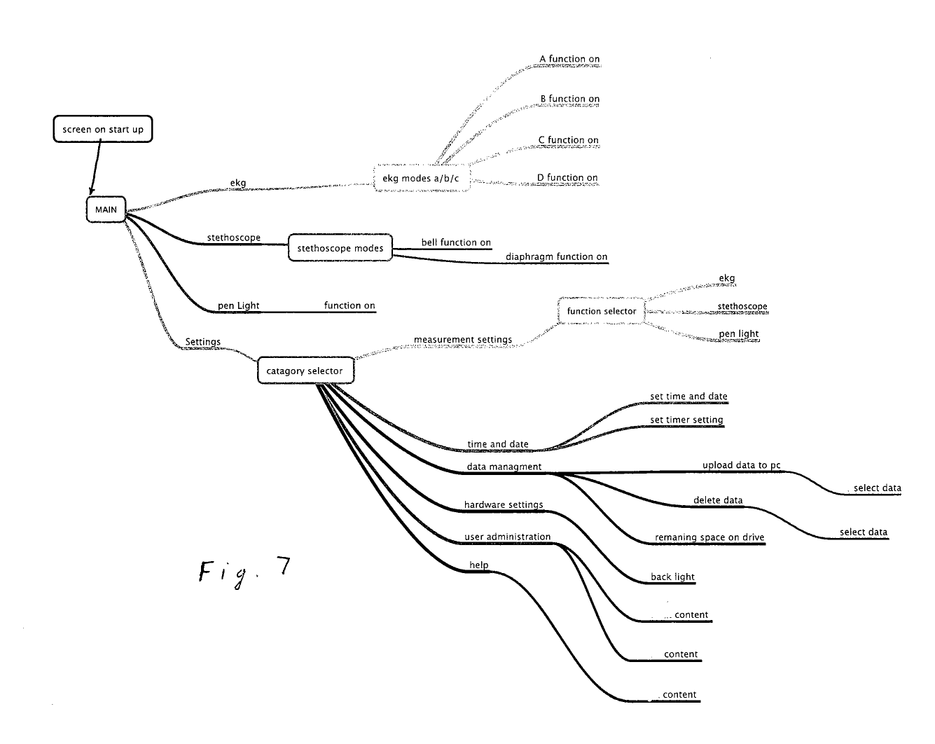

Apparatus 10 includes a second body or portion 14 having a connecting junction to portion 11. Within the second portion 14, the sensor housing of a pulse oximeter sensor 110 (FIG. 5) may be placed. A power recharging port 21 is disclosed. Additional connection ports may be provided for connection to the sensor disposed in pulse oximeter sensor housing (not shown). The data may also be sent to a pulse oximeter CPU 110 mounted on a printed circuit board 250 (FIG. 7B), as discussed below. The second portion 14 advantageously includes electrodes 44 and 46, which are held by a patient, to generate and ECG. An additional set of electrodes may be placed on the opposite side of the second portion, thus facilitating holding by the patient.

Apparatus 10 includes a third portion 20 having a headset 36 to allow the user of apparatus 10 to listen to sounds from a body acquired by diaphragm 22 and a microphone 112, and an audio microphone at 38 to pick up sounds from the user (the user's voice) to allow the user to communicate via voice/data cellular and internet connection apparatus 120, as more fully described with respect to FIG. 5 below. Earpieces 50, which are described in detail below, provide a comfortable fit to the ear of the medical professional using the third portion 20. Earpieces 50 are at the end of tubes 51, which conduct sounds to earpieces 50. Tubes 51, which are generally formed of a high quality metal, may be flexed outward. Arrows A represent the motion of tubes 51, which when not acted upon by an external force, will return to a closed position, as represented in FIG. 1.

Referring to FIG. 5, the first portion or head 11 of apparatus 10 includes a processor in the form of a main CPU/memory 100, powered by a rechargeable battery 102, which may be charged via a USB connector in a manner well know in the art and outlet provided at part #2 (accessible when part #3, headset is removed). The display screen 15, which may be a color display, with touch screen features, displays images generated by main CPU/memory 100. Data entry may be accomplished via key data entry buttons 12, 17, as described above. As also described above, a set of converging lights 40, 42 may also be powered by battery 102. A camera video lens input from a video camera on first portion or head 11 may be provided to main CPU/memory 100. The converging lights 40, 42 may be used to illuminate a region of the body that is to be photographed by the camera, as described above. Other sensors that may be included on head 11 are a sonogram probe and EKG/ECG electrodes 108. A pulse oximeter CPU mounted on a printed circuit board 110 within head 11 also can send data to CPU/memory 100. A sound detector microphone 112 also can send data to CPU/memory 100. A microphone/sound CPU 114 can assist in processing sound information.

Head 11 may also include a wireless transmitter/receiver 116 for receiving and transmitting data, especially the data obtained from the sensors described above. An alphanumeric wireless text/pager 118 can be integrated into head 11 to provide appropriate alerts and pager messages to a user of apparatus 10, thus eliminating the need to have a separate pager. The pager electrical components, including receiver maybe relegated to part #2 and data and input functions accessed from part #1. Likewise the pager maybe provided as an integrated accessory within part #3 (headset). In addition, provisions may be made for a voice/data cellular and internet connection apparatus 120 so that those functions are also available to a user of apparatus 10.

Portion 14 of apparatus 10 includes electromagnetic antennas 122 for transmitting and receiving signals to implement the various functions of apparatus 10 requiring such communication. In addition, hardware such as the pulse oximeter sensor (Spo2) 124 and left and right ECG/EKG leads 126 may be associated with portion 14. In this regard, reference is made to the teachings of U.S. Pat. No. 7,300,406, with respect to EKG leads, a compartment in portion 14 for such leads, and doors to cover such compartment, in which the leads can be stored when not in use.

Portion 14 may also be equipped with USB input/output ports for the acquisition of Spo2 and other data, and to generally facilitate data exchange between portion 14 and additional systems/devices/portable instruments that are interchangeable with the headset. Portion 14 is also includes digital audio connectors 130 for connection of audio signals acquired during examination of a body with third portion 20 of apparatus 10 and a loud speaker 48.

Third portion 20 of apparatus 10 includes a headset 36 to allow the user of apparatus 10 to listen to sounds from a body acquired by apparatus 10, and a microphone 38 pick up sounds from the user (the user's voice) to allow the user to communicate via voice/data cellular and internet connection apparatus 120 or to record comments and observations in the memory of main CPU/memory 100.

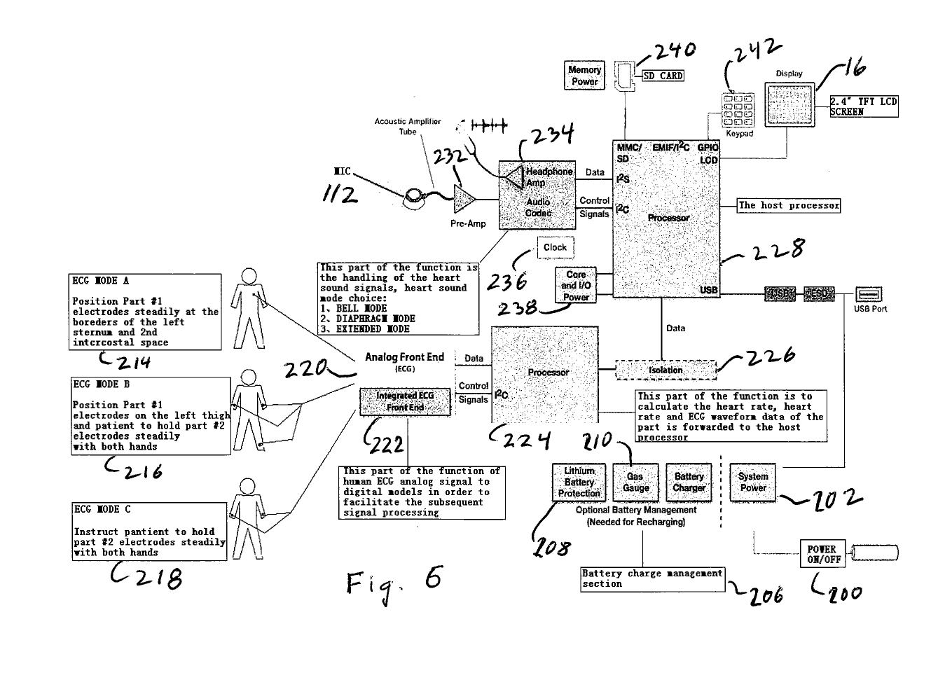

FIG. 6 illustrates a functional block diagram of the major electrical components of the illustrated embodiment. An on/off switch 200 powers up the power system 202, to provide power for the illustrated components, including a USB port 204. In view of the apparatus being portable, a battery charge management section 206 includes a lithium battery protection circuit 208, a gas pressure sensor of gage 210, and a battery charger 212.

Three different ECG modes A, B and C (denoted by reference numerals 214, 216 and 218, respectively), including modes where the patient holds electrodes on part 14, can feed signals to analog front end 220. An integrated portion 222 of the front end 220, converts the analog signals using digital models, to provide control signals to a processor 224. Processor 224 also receives data from analog front end 220. Processor 224 calculates heart rate, heart rate and ECG waveform data and sends it through a safety isolator 226 to a host processor 228. In one ECG mode, the patient will hold the electrodes of parts #2 with the right and left hands to obtain Lead 1 ECG tracing. In another mode, the patient will hold the electrodes of part #2 (front and back, using right and left hands respectively), while the electrodes of the first portion is positioned on the left leg (to create Einthoven triangle formation). In this configuration, the detection of all 6 limb leads will be acquired (namely leads I, II, III, Avf, AvR, and AvL). When the electrodes of the first portion is positioned on the patients' chest, at least one of the aforementioned 6 total number of leads will be obtained. The 4 electrodes found on the first portion will recreate a miniature Einthoven's triangle over the patient's heart. Results may vary using this method, due to patient's body mass, hair or operator's technique.

An acoustic microphone 230 provide signals to a pre-amplifier 232, which in turn provides signals an audio amplifier 234, which depending upon the software selection can treat acoustic signals in the towel mode, the diaphragm mode, or extended mode. Data and control signals from audio amplifier to 34 are also supplied as inputs to processor 228. Processor 228 has associated with it a clock 236, and a core 238, and may provide power to various input and output devices. While processor to 24 will generally have local memory, data stored on an SD card 240 may be utilized by processor 228. Further, processor 228 will be interfaced to a keyboard 242 (or the more limited set of keys or entry buttons 12, 13, 16 and 17 of FIG. 1), as well as display screen 16 (also FIG. 1).

A variety of data may be shown on the display screen 16. Selections may include the EKG trace, the audio waveform of the heartbeat, the heart rate, and patient data such as social security number, date and age. Depending upon what other sensors are included in apparatus 10 (interchangeable with headset), other data that may be displayed may include blood oxygen level, an ultrasonic image, or a part of the body illuminated by lights 40 and 42.

FIG. 7 illustrates one possible approach for the arrangement of the images and data, and the manner in which one display screen may be accessed from another display screen. Generally, the manner in which this occurs will depend on programming of processor 228 with appropriate firmware.

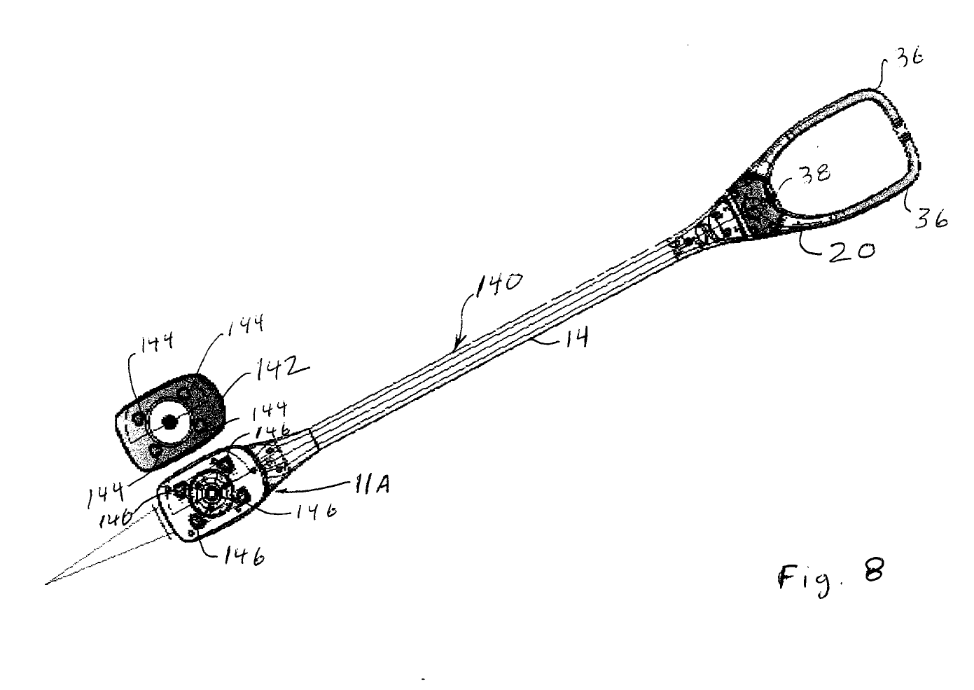

FIG. 8 is an embodiment 140 having a removable plate or module 142 which may have affixed thereto a set of electrodes 144 in electrical contact with a respective, underlying set of electrical contacts 146 on a surface of the head 11A of embodiment 140 that is covered by module 142 when the embodiment is in use. Module 142 may be configured to snap into the head 11A of embodiment 140, so that the undersides of electrodes 144 are in contact with respective surfaces of electrical contacts 146. Other attachment means may be used to releasably affix module 142 to head 11A, such as screws or various mechanical release mechanisms.

In view of module 142 being removeably connected to head 11A, different modules 142, having somewhat different electrode configurations, may be snapped into place, thus allowing the user to choose from among the various configurations, as may be preferred in different situations or for different patients, or simply to replace the module if the electrodes 144 become worn or for some reason not usable for further examination.

Other sensors may be affixed to module 142 such as temperature, pulse/Doppler probes and oxygen probes.

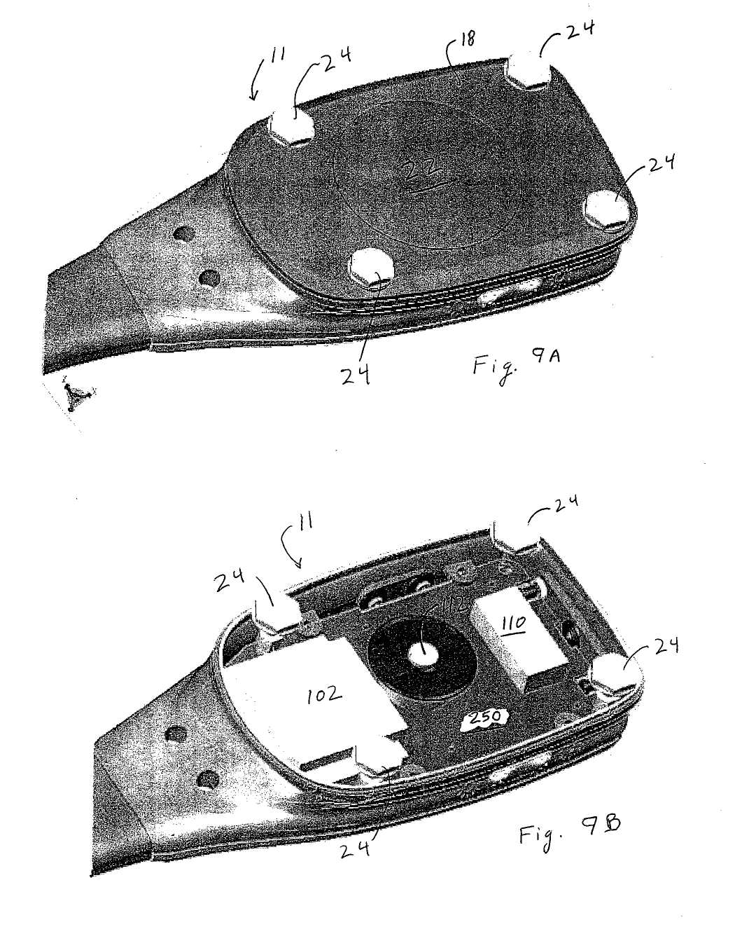

Referring to FIGS. 9A and 9B, each of ECG electrodes 24 may be in the form of a removable and replaceable screw with, for example, a hexagonal head. As illustrated in FIG. 7A, which shows a cover removed to illustrate internal components, such as microphone 112, a circuit board 250 and battery 102. While screws 24 may be formed of copper they are advantageously plated with silver or a silver compound such as solar chlorides, to facilitate the best electrical contact with the skin of a patient. The electrodes may also be made of a pure element such as silver. However, over a period the silver or silver compound coating may deteriorate or dissipate with use. Thus, a tool (not shown) may be provided to unscrew electrodes 24 by engaging the hexagonal head. A replacement kit consisting of four screws and the tool may be provided or sold separately, to replace the electrodes 24.

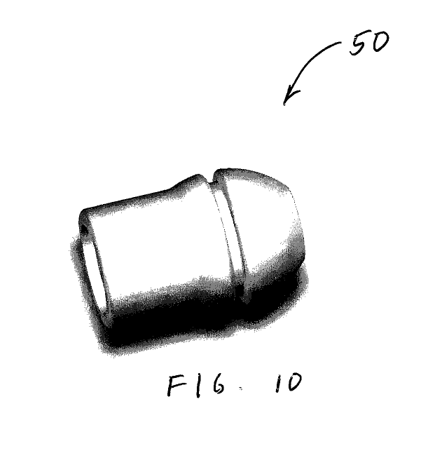

FIG. 10 is a perspective view of an earpiece 50.

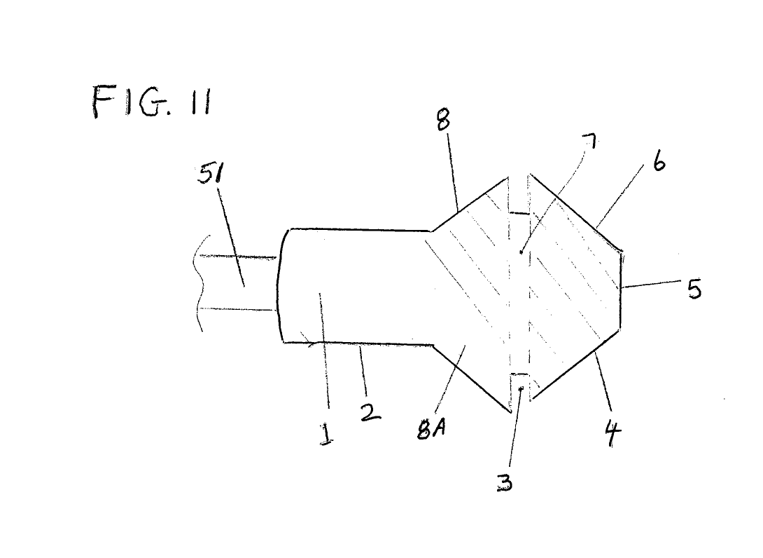

Referring to FIG. 11, when the earpiece is subjected to appropriate force, usually less than one pound, as exerted by a spring loaded stethoscope, the force will be redirected toward the crumble zone and collapsible zones 3 and 7 respectively. Most of the exerted pressure will be absorbed in this region, distorting and providing counter force to secure zone 3 over the opening of an ear canal tightly and comfortably. Zone 2 in FIG. 11 is denoted as area 3, which compresses under external force as exerted from a spring-loaded stethoscope. Area 7 may be corrugated or constructed of resilient material in order to absorb the force uniformly in a relief area. The zone 3 outer ring, region 4, is preferably constructed of suitable material such as silicone with less thickness and hardness than zones 1 and 2. Zone 3 will come in direct contact with the ear, therefore it should be of suitable comfortable material that will not promote irritation or the like.

Zone 1 may be furthest away from the ear canal opening, and therefore may be constructed of more resilient, thicker material, thereby providing anchoring and structural support for the other two parts.

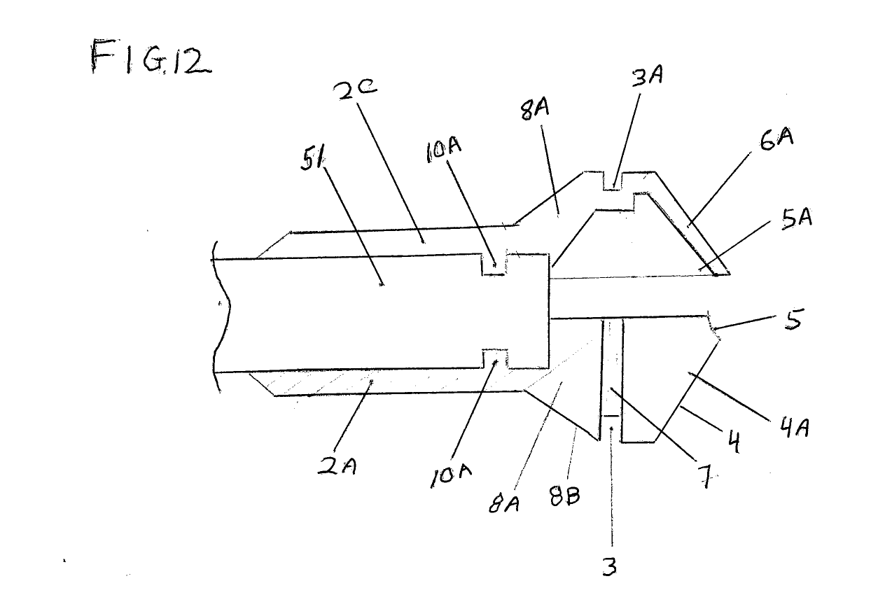

With reference to FIG. 12, Zone 1 may have an anchoring protrusion 10A that allows it to be removably fixable over the stethoscope's tube 51. A flexible arm/crumble zone 3 and 3A is further illustrated in detail. The terminal aspect of the earpiece 6A (internal) and 4A (external) is of lesser thickness and causes minimal distortion and secure fitting over the opening of an ear canal.

Other features and uses of the apparatus described herein may include:

1. Examining an ear canal and performing a fundoscopic exam, using photos and video means that are interchangeable with the headset of part #3.

2. A signature placement area on the apparatus to allow inscription such as a signature placement or personalize markings.

3. Recharging of the internal battery of the apparatus using induction, conduction method or USB port (located on any one of the first, second or third portions) as well as conventional power outlet means using a voltage converter.

4. The first portion further comprises a ‘touch screen’ OLED display visual indication/viewing which is representative of at least one of the EKG signals, photo, audio, text message or video.

5. An electronic stethoscope with audio/video storage, conferencing, acquisition and playback means in the form of music, spoken words, lectures, video, text etc.

6. An electronic stethoscope with innate audio speakers near the earpieces, which may broadcast sounds to bystanders and patients.

7. A stethoscope device with a security function in the form of speech recognition.

8. A stethoscope device with security function in the form of finger print identification means.

9. A stethoscope device with a Doppler probe which measures the pulse pressure and converts same to blood pressure expressed as mm/hg for diastolic and systolic parameters.

10. A stethoscope device with an infrared light source.

11. A stethoscope with a projector lens to broadcast audio/video on a surface.

12. An apparatus as described herein with a Snellen eye chart in digital form, displayable on the screen of the first part.

13. A apparatus as described herein with a ruler measuring reference on at least the second part.

It will be understood that the availability of a variety of sensors, connections to infrastructure such as the interne or wireless telephone networks, and the on-board data processing capability allow the apparatus to be used efficiently in a large number of ways to assist in the diagnosis of the condition of a patient in a mobile or within an institution. For example sounds and video information may be transmitted to distant locations to assist in diagnosis. Experts in a diagnosed condition can be contacted by telephone and data transmitted to them for advice on appropriate treatments. Additional uses can be developed as the versatility and convenience of the apparatus are explored by users.

It should be understood that the foregoing description is only illustrative of the invention. Various alternatives and modifications can be devised by those skilled in the art without departing from the invention. Accordingly, the present invention is intended to embrace all such alternatives, modifications and variances which fall within the scope of the appended claims.

1. An apparatus for determining a condition of a living body, comprising:
a first portion for acquiring acoustic signals from said body, said first potion having removeable electrodes for acquiring electrical signals from said body;
a second portion including an elongate housing having conduits for conducting signals from said first portion; and
a third portion removeably connectable to said second portion, said third portion including at least one interchangeable instrument.

2. The apparatus of claim 1, wherein the interchangeable instruments include at least one of a headset, a speaker, and a medical probe.

3. The apparatus of claim 1, wherein the interchangeable instruments include at least one medical probe selected from the group comprising a pulse oximeter, an ultrasound transducer, a camera, an otoscope and an opthalmoscope.

4. The apparatus of claim 1, further comprising a display on the first portion for displaying acoustic signal or electrical signals from said first portion, or signals from said interchangeable instrument.

5. The apparatus of claim 1, wherein said third portion is releasably connected to said second portion, further comprising a communication link between said second portion and said third portion for transferring signals when said third portion is not connected to said second portion.

6. The apparatus of claim 5, wherein said communication link is a broadband communications link.

7. The apparatus of claim 5, wherein said communication link is a wireless communication link.

8. The apparatus of claim 1, wherein said first portion further comprises a display for providing a visual indication representative of images from a camera lens disposed on said first or second portions.

9. The apparatus of claim 8, in combination with an external display coupled to said apparatus with a broadband wireless real-time connection so that said external display wirelessly receives at least one of audio, photo, text and video signals.

10. The apparatus of claim 1, wherein said first and second portions further comprises a camera lens for photo and video recording from at least a portion of said body.

11. The apparatus of claim 10, in combination with an external display coupled to said apparatus with a broadband wireless real-time connection so that said external display wirelessly receives at least one of acoustic signals, electrical signals, and signals acquired by an ultrasonic transducer, ECG electrodes, microphone, and camera lens.

12. The apparatus of claim 1, wherein said second portion is configured with an elongate cavity or body, having a width that is wider than its height.

13. The apparatus of claim 1 wherein a pulse oximeter is housed in a cavity of the second portion or connected via a connecting port to said second portion.

14. The apparatus of claim 1, wherein said electrodes are in the form of screws that are releasably connected to said apparatus.

15. The apparatus of claim 1, wherein said electrodes are formed of copper, and a surface of said electrodes for contacting a body is coated with silver or silver chloride.

16. The apparatus of claim 1, further comprising a lens for photos and video recording located on first, second or third portion.

17. The apparatus of claim 1 wherein the second portion has a width that is wider than its height giving it a flat shape and a width to height ratio of one or greater than one.

18. The apparatus of claim 1, with a first part being generally square or rectangular at its base and top section with a length to width ratio of one or greater than one.

19. The apparatus of claim 1, with a first portion with a width to length ratio of one or less than one.

20. An apparatus of claim 1, with a touch screen data entry user inter-phase on a first part and user interface buttons located on sides of the first part.

21. The apparatus of claim 1 with wireless phone capability, whereby the third portion is fitted with a microphone and listening headset for communicating with a user on a telephone device or another similar stethoscope device.

22. The apparatus of claim 1, further comprising at least one adjustable light source on said first portion, whereby two or more light sources of adjustable brightness converge at a focal point, forward of the device.

23. The apparatus of claim 1, wherein said first portion is releasably connected to said second portion, and said second portion is releasably connected to said third portion.

24. The apparatus of claim 1, wherein said first portion comprises a module which removeably attaches to said first portion, said module being for supporting said electrodes for contact with said body.
